SEQUENCE LISTING (14 sequences)

SEQ ID NO: 1 - DNA, 20 bp, synthetic construct
  misc_feature 1..20: note = artificial primer
  source 1..20: mol_type = other DNA
atcttttccgactcattgtc

SEQ ID NO: 2 - DNA, 20 bp, synthetic construct
  misc_feature 1..20: note = artificial primer
  source 1..20: mol_type = other DNA
gattttctccagcaattcgg

SEQ ID NO: 3 - DNA, 20 bp, synthetic construct
  misc_feature 1..20: note = artificial primer
  source 1..20: mol_type = other DNA
ccgaattgctggagaaaatc

SEQ ID NO: 4 - DNA, 20 bp, synthetic construct
  misc_feature 1..20: note = artificial primer
  source 1..20: mol_type = other DNA
cgaatgaaggttattgaacg

SEQ ID NO: 5 - protein, 60 aa, Streptococcus salivarius
MAKNTSRPEIDSLSFEVENQELSGKSGSGWFTAVQLTLAGRCGRWFTGSFECTTNNVKCG

SEQ ID NO: 6 - protein, 60 aa, Streptococcus salivarius
MRKNNNRKEIDTLDFEVKNQELSGKSGSGWFTAVQLTLAGRCGRWFTGSYECTTNNVKCG

SEQ ID NO: 7 - protein, 60 aa, Streptococcus salivarius
MKSKKVNTEIDTLEFEIDNQELNGTSGSGWWYTAFKMTLAGRCGLCFTCSYECTTNNVHC

SEQ ID NO: 8 - protein, 58 aa, Streptococcus salivarius
MKQDNFEIDSLDYEINSQELNGKSAAGWSTAVRLTVQGRCGWWFTHSYECTSPNVRCG

SEQ ID NO: 9 - protein, 158 aa, Streptococcus salivarius
MIGKNIKSLRKTHDLTQPEFARIVGISRNSLSRYENGTSSVSTELIDIICQKFNVSYVDIVGEDKMLNPVEDYELTLKIEIVKERGANLLSRLYRYQDRQGISIDEESNPWILMSDDLSDLIHTNIYLVETFDEIERYSGYLDGIERMLEISEKRMVA

SEQ ID NO: 10 - protein, 256 aa, Streptococcus salivarius
MEIQDYTDSEFKHALARNLRSLTRGKKSSKQPIAILLGGQSGAGKTTIHRIKQKEFQGNIVIIDGDSFRSQHPHYLELQQEYGKDSVEYTKDFAGKMVESLVTKLSSLGYNLLIEGTLRTVDIPKKTAQLLKNKGYEVQLAIIATKPELSYLSTLIRYEELYAINPNQARATPKEHHDFIVNHLVDNTRQLEELSIFERIQIYQRDRSCVYDSKENTTSAATVLHDLLFGEWNQVEKEMLKSGEERLKDLTNRNGG

SEQ ID NO: 11 - protein, 330 aa, Streptococcus salivarius
MGIGIIIASHGKFAEGIHQSGSMIFGDQEKVQVVTFMPSEGPDDLYAHFNDAIAQFDADDEILVLADLWSGSPFNQASRIAGENPDRKIAIITGLNLPMLIQAYTERMMDANATAEQVAANIIKEAKGGIKALPEELNPAEETTAAPVEAAAPQGAIPEGTVIGDGKLKINLARLDTRLLHGQVATNWTPASKADRIIVASDDVAKDELRKELIKQAAPNGVKANVVPIQKLIDASKDPRFGNTHALILFETVQDALRAIEGGVPIKELNVGSMAHSTGKTMVNNVLSMDKDDVACFEKLRDLGVEFDVRKVPNDSKKDLFDLIKKANVQ

SEQ ID NO: 12 - protein, 271 aa, Streptococcus salivarius
MSDMSIISAILVVVVAFLAGLEGILDQFQFHQPLVACTLIGAATGNLTAGIMLGGSLQMIALAWANIGAAVAPDAALASVAAAIILVKGGNFTTEGIGVATATAIPLAVAGLFLTMLVRTASVALVHAADKAAESGNIAGVERAHYLALLLQGLRIAVPAALLLAIPAQSVQHALGLMPDWLNHGMVVGGGMVVAVGYAMVINMMATREVWPFFAIGFAFAAISQLTLIALGAIGVAIAFIYLNLSKQGGGNGGGTSSGSGDPIGDILEDY

SEQ ID NO: 13 - DNA, 20 bp, synthetic construct
  misc_feature 1..20: note = artificial primer
  source 1..20: mol_type = other DNA
ctctcagctaatcacaactc

SEQ ID NO: 14 - DNA, 20 bp, synthetic construct
  misc_feature 1..20: note = artificial primer
  source 1..20: mol_type = other DNA
gtagtcttccaagatatcgc

(19) United States
(12) Patent Application Publication
(10) Pub. No.: US 2023/0381249 A1
(43) Pub. Date: Nov. 30, 2023

(54) PROBIOTIC STRAIN FOR COUNTERING CARIES PATHOGENS

(71) Applicant: The Governing Council of the University of Toronto, Toronto (CA)

(72) Inventors: Celine M. LEVESQUE, Toronto (CA); Siew-Ging GONG, Toronto (CA)

(21) Appl. No.: 18/167,689

(22) Filed: Feb. 10, 2023

Related U.S. Application Data
(60) Provisional application No. 63/308,833, filed on Feb. 10, 2022.

(51) Int. Cl. A61K 35/744; A61K 9/00; A61K 9/50; A61P 1/02; A61P 31/04

(52) U.S. Cl. CPC A61K 35/744; A61K 9/0053; A61K 9/5036; A61P 1/02; A61P 31/04

(57) ABSTRACT

A strain of Streptococcus salivarius, probiotic mixtures and compositions have improved antimicrobial activity against Streptococcus mutans. Also provided are methods of preventing caries caused by Streptococcus mutans, comprising administering the probiotic mixture or the composition.

### CROSS-REFERENCE TO RELATED APPLICATION

This application claims the benefits of priority to U.S. Provisional Patent Application No. 63/308,833, filed Feb. 10, 2022, titled PROBIOTIC STRAIN FOR COUNTERING CARIES PATHOGENS, the contents of which are hereby expressly incorporated into the present application by reference in their entirety.

### FIELD OF THE INVENTION

The invention relates to an isolated strain of Streptococcus salivarius, and certain protein products of same, and their use in countering caries pathogens.

### BACKGROUND OF THE INVENTION

The mouth is the portal of entry to the digestive system and nutrition of vital importance to our health. The mouth is also a major gateway to infectious diseases. It is home to over 700 bacterial species occupying multiple niches, called the oral microbiome. Under normal circumstances, the oral microbiome remains relatively stable (microbial homeostasis) contributing to oral health. However, with continued high intake of free sugars, inadequate exposure to fluoride and without regular plaque removal, tooth structures are destroyed, resulting in development of cavities (tooth decay) and pain due to these cavities. Dental cavities or caries has major impacts on oral health-related quality of life, and, in the advanced stage, tooth loss and systemic infections. Dental caries is the most common infectious diseases affecting humans. In fact, almost half of the world's population is affected by caries, making it the most prevalent of all health conditions.

It has been known for more than a century that caries is a bacterially mediated process. These bacteria are found in the dental plaque biofilm that forms within minutes to hours after a professional dental cleaning. Under normal circumstances, the dental biofilm remains relatively stable in a microbial homeostasis that contributes to dental health. When microbial dysbiosis occurs, e.g., increased colonization by highly acidogenic and aciduric mutans streptococci (MS; mainly of the species Streptococcus mutons and Streptococcus sobrinus), often in synergy with other bacteria and fungi, e.g., Candida albicans, caries occurs. MS bacteria are embedded in an extracellular matrix rich in glucan and fructan derived from the metabolism of dietary sucrose. The adherent plaque biofilm provides thus an ecological niche for cariogenic bacteria to thrive and produce various organic acids. The localized metabolic production of acids from MS decays the tooth through the demineralization of calcium and phosphorous from the enamel and dentin over several months, resulting in caries lesions.

Probiotic bacteria have become increasingly popular during the last decade because of their health-beneficial effects. They have been widely marketed and consumed, mostly as dietary supplements or functional foods to enhance or restore balance to the gut microbiome. Oral probiotics against dental caries represent new therapeutic approaches based on restoring the microbial ecological balance in the oral cavity. Although probiotics containing beneficial bacteria have been used to prevent or combat diseases caused by pathogens, probiotics against dental caries have not worked well since many of these probiotic strains do not persist in the oral cavity. However, such strains and probiotic approaches do exist, and are disclosed, for example, in U.S. Pat. No. 10,857,090, U.S. provisional application 61/674,390, and others, all incorporated by reference.

The bacterial species Streptococcus salivarius is a pioneer colonizer of the mouth and a predominant member of the native oral microbiome that persists throughout the human lifespan. S. salivarius has an exclusive and intimate association with humans. It normally colonizes the buccal epithelium and is a resident of the tongue dorsum. In saliva, it is typically present at levels of up to 10 million colony-forming units per milliliter. S. salivarius has emerged as an important source of safe and efficacious probiotics capable of fostering a more balanced, health-associated oral microbiome. Some strains of S. salivarius produce a variety of lantibiotic bacteriocins that are usually encoded on transmissible megaplasmids. These antimicrobial peptides contain the non-genetically encoded amino acids lanthionine and/or mehyllanthionine and various other highly modified amino acids, such as dehydroalanine and dehydrobutyrine. Lantibiotics are produced by, and primarily act upon, Gram-positive bacteria. They kill bacteria rapidly and are active at very low concentrations. Their inability to kill Gram-negative bacteria results from their relatively large size which prevents them from penetrating the outer membrane. Few S. salivarius strains have been reported to produce lantibiotics capable of interfering with the growth of oral pathogens. BLIS K12®, the first probiotic specifically developed for oral health, has been used as an oral probiotic against group A streptococci pharyngitis (sore throat) and halitosis (bad breath) without safety issues for many years. Indeed, S. salivarius KU strain received FDA generally recognized as safe (GRAS) status in the U.S. in 2011. In 2015, Health Canada approved the oral probiotic BLIS K12® for use in supplement products. K12 strain produces two lantibiotics, salivaricin A2 and salivaricin B encoded on two adjacent loci on a 190-kb megaplasmid. The inhibitory spectrum of K12 encompasses Streptococcus pyogenes and some of the key anaerobes that have been implicated in halitosis. K12 also inhibits the growth of pathogens involved in the etiopathogenesis of acute otitis media. The second developed anti-halitosis probiotic was S. salivarius M18 (BLIS M18®) which was also shown to exhibit inhibitory potential against periodontal pathogens. BLIS M18® has been developed as an oral probiotic to help prevent gingivitis and dental plaque buildup. The need, therefore, exists for a new generation of S. salivarius strains specifically targeting caries pathogens to be developed as oral care probiotics for the prevention of dental caries.

In the oral cavity, sugars are the only usable source of energy for most oral streptococci. Thus, S. salivarius possesses high-affinity sugar transport systems to compete successfully and persist in the oral cavity. Transport of sugars in streptococci is mediated principally by the phosphoenolpyruvate:sugar phosphotransferase system (PTS). In most streptococci, glucose (a commonly used sugar) is transported by the PTS:glucose/mannose, a multienzymatic system that sequentially catalyzes the transport and phosphorylation of glucose, mannose, and fructose. In addition to its role in the uptake of usable source of energy, the PTS:glucose/mannose is also involved in the transport of 2-deoxyglucose (2-DG), an analog of glucose that is lethal for oral streptococci. The toxicity of this analog is caused by the establishment of a futile energy cycle that leads to the dissipation of phosphoenolpyruvate and ATP, causing bacterial cell death. In S. salivarius, the PTS:glucose/mannose is composed of a tetracistronic operon that is constitutively expressed. The operon is composed of four genes, manL, manM, manN, and manO (FIG. 1). It has been shown that mutations affecting the expression of the PTS:glucose/mannose components, especially, manL, have pleiotropic consequences on the expression of a wide variety of membrane and cytoplasmic proteins. Several reports have also implicated the PTS:glucose/mannose in the control of sugar utilization in oral streptococci such as the control of lactose utilization.

Probiotic compositions are known; a probiotic adds beneficial cultures to populations of microbial flora. There is a need for improved formulations of probiotic compositions, particularly formulations that result in one or more of improved oral health, improved oral colonization, improved efficacy, as well as methods of administration or uses thereof.

### SUMMARY OF THE INVENTION

According to one aspect of the present invention is provided an isolated strain of Streptococcus salivarius having antimicrobial activity against Streptococcus mutons (S. mutons).

In certain embodiments, the strain has stronger adhesion property to biotic and abiotic surfaces.

In certain embodiments, the strain has the ability to form higher biomass as compared to other known strains.

In certain embodiments, the isolated strain of Streptococcus salivarius has resistance to deoxyglucose metabolite and/or deletion of manL and manM genes.

In certain embodiments, the strain expresses Pep1 having SEQ ID NO.: 5, Pep2 having SEQ ID NO.: 6, Pep3 having SEQ ID NO.: 7, and Pep 4 having SEQ ID NO.: 8.

In certain embodiments, the isolated strain of Streptococcus salivarius expresses the Pep1, Pep2, Pep3 and Pep4 as a multipeptide protein.

In certain embodiments, the isolated strain of Streptococcus salivarius has a 183,700 base pair megaplasmid encoding Pep1, Pep2, Pep3, and Pep4.

In certain embodiments, the 183,700 base pair megaplasmid further expresses two ABC transporter ATPase proteins, an ABC transporter permease protein, a histidine kinase, a response regulator, a lantibiotic synthesis protein, and an ABC-type bacteriocin/lantibiotic exporter.

In certain embodiments, the isolated strain of Streptococcus salivarius has S. mutons inhibitory or toxicity activity.

In certain embodiments, the S. mutons inhibitory or toxicity activity occurs in a high sugar environment.

In certain embodiments, the isolated strain of Streptococcus salivarius has inhibitory or toxicity activity against S. mutons serotype c, e, f, k or any combination thereof.

In certain embodiments, the isolated strain of Streptococcus salivarius has caries prevention activity.

In certain embodiments, the isolated strain of Streptococcus salivarius has inhibiting activity against streptococcal throat infections.

In certain embodiments, the isolated strain of Streptococcus salivarius has inhibiting activity against periodontal diseases.

According to a further aspect of the present invention is provided an isolated peptide having at least 90% amino acid similarity or identity to the sequence of SEQ ID No.: 5, SEQ ID NO.: 6, SEQ ID NO.: 7, or SEQ ID NO.: 8 and antimicrobial activity against S. mutons.

According to a further aspect of the present invention is provided an isolated peptide Pep1, Pep2, Pep3, or Pep4, having a sequence of SEQ ID NO.:5, SEQ ID NO.:6, SEQ ID NO.:7, or SEQ ID NO.: 8, respectively.

According to a further aspect of the present invention is provided an isolated multipeptide protein comprising peptide Pep1 having a sequence of SEQ ID NO.: 5, peptide Pep2 having a sequence of SEQ ID NO.: 6, peptide Pep3 having a sequence of SEQ ID NO.: 7, and peptide Pep4 having a sequence of SEQ ID NO.: 8.

According to a further aspect of the present invention is provided a probiotic mixture comprising the isolated strain, the isolated peptide, and/or the isolated multipeptide protein as herein described.

According to a further aspect of the present invention is provided a composition for oral administration for preventing caries caused by cariogenic bacteria such as S. mutons, comprising the isolated strain, the isolated peptide, the isolated multipeptide protein, and/or the probiotic mixture as herein described.

According to a further aspect of the present invention is provided a composition for oral administration for preventing or inhibiting biofilms formed by cariogenic bacteria and fungi such as S. mutons, human dental plaque and Candida albicans, comprising the isolated strain, the isolated peptide, the isolated multipeptide protein, and/or the probiotic mixture as herein described.

According to a further aspect of the present invention is provided a composition for oral administration for prevention or treatment of streptococcal throat infections, comprising the isolated strain, the isolated peptide, the isolated multipeptide protein, and/or the probiotic mixture as herein described.

According to a further aspect of the present invention is provided a composition for oral administration for prevention or treatment of periodontal diseases comprising the isolated strain, the isolated peptide, the isolated multipeptide, and/or the probiotic mixture as herein described.

In certain embodiments, the probiotic mixture, or composition, is in the form of a lollipop, a candy such as a gummy candy, a lozenge, a mouth wash, a liquid rinse, a toothpaste, a drink, a chewable, a tooth varnish or coating, a lyophilized probiotic powder, or a food.

In certain embodiments, the probiotic mixture, or composition, is in a polysaccharide encapsulated form. In certain embodiments, the polysaccharide encapsulation is an alginate polysaccharide. In certain embodiments, the polysaccharide encapsulation is a chitosan coated alginate polysaccharide, or a combination chitosan-alginate polysaccharide.

According to a further aspect of the present invention is provided a method of preventing caries caused by S. mutons, comprising administering the herein described probiotic mixture or composition.

According to a further aspect of the present invention is provided a method of treating or preventing dental caries, comprising administering the herein described probiotic mixture or composition.

According to a further aspect of the present invention is provided a method of treating or preventing streptococcal throat infections, comprising administering the herein described probiotic mixture or composition.

According to a further aspect of the present invention is provided a method of manufacturing an isolated peptide, or an isolated multipeptide protein, as herein described, comprising growing the isolated strain as herein described in a bacterial growth medium in vitro, then isolating the protein therefrom.

According to a further aspect of the present invention is provided a method of manufacturing a herein described isolated peptide, comprising: amplifying by PCR a DNA sequence capable of translation into amino acid sequence of SEQ ID No.: 5, SEQ ID NO.: 6, SEQ ID NO.: 7, or SEQ ID NO.: 8 or an amino acid sequence having at least 90% amino acid similarity or identity to the sequence of SEQ ID No.: 5, SEQ ID NO.: 6, SEQ ID NO.: 7, or SEQ ID NO.: 8; inserting a DNA sequence capable of translation into an amino acid sequence of SEQ ID No.: 5, SEQ ID NO.: 6, SEQ ID NO.: 7, or SEQ ID NO.: 8 or an amino acid sequence having at least 90% amino acid similarity or identity to the sequence of SEQ ID No.: 5, SEQ ID NO.: 6, SEQ ID NO.: 7, or SEQ ID NO.: 8 into a DNA vector containing a promoter region for expressing said DNA sequence; inserting said PCR amplified DNA or DNA vector containing the DNA sequence into a bacterium; growing the bacteria in vitro in a bacterial growth medium, to express the amino acid sequence; isolating the protein from the grown bacteria or a supernatant of the bacterial growth medium.

According to a further aspect of the present invention is provided a method of manufacturing an isolated multipeptide protein as herein described, comprising: amplifying by PCR a DNA sequence capable of translation into an amino acid sequence of SEQ ID No.: 5, SEQ ID NO.: 6, SEQ ID NO.: 7, and SEQ ID NO.: 8 or an amino acid sequence having at least 90% amino acid similarity or identity to the sequence of SEQ ID No.: 5, SEQ ID NO.: 6, SEQ ID NO.: 7, and SEQ ID NO.: 8; inserting DNA sequences capable of translation into an amino acid sequence of SEQ ID No.: 5, SEQ ID NO.: 6, SEQ ID NO.: 7, and SEQ ID NO.: 8 or an amino acid sequence having at least 90% amino acid similarity or identity to the sequence of SEQ ID No.: 5, SEQ ID NO.: 6, SEQ ID NO.: 7, and SEQ ID NO.: 8 into a DNA vector containing a promoter region for expressing said DNA sequences; inserting said PCR amplified DNA or DNA vector containing the DNA sequences into a bacterium; growing the bacteria in vitro in a bacterial growth medium, to express the amino acid sequences; isolating the isolated multipeptide protein which spontaneously forms from said expressed amino acid sequences from the grown bacteria or a supernatant of the bacterial growth medium.

### BRIEF DESCRIPTION OF FIGURES

FIG. 1 shows a schematic representation of the locus encoding the PTS:glucose/mannose in S. salivarius. Arrows indicate the PCR primers used for the molecular typing. The digoxigenin-labeled DNA probes used for the detection of manL and manM genes by Southern blotting are indicated as hatched boxes, manL; IIAB enzyme, manM; IIC protein; manN; IID protein; IID protein; manO; putative regulator ManO. The primer pair CMT-1542/1556 was used to detect the 5′ end portion of manL gene while the primer pair CMT-1557/CMT1543 was used to detect the 3′ end portion of the gene.

FIG. 2 shows detection of manL and manM genes by Southern blotting. Total genomic DNA of S. salivarius was digested with EcoRI, HindIII or XbaI. WT: LAB813, used as control; V6: variant VAR6. The manL-specific and manM DNA probes are indicated by hatched boxes below the corresponding ORF in FIG. 1.

FIG. 3 shows microscopic observation of S. salivarius parental strain (A) and its variant VAR6 (B).

VAR6 possesses the ability to grow in the presence of 2-DG, an inhibitor of glycolysis and to form microcolonies and large cellular aggregates (B) in contrast to the typical individual cocci or small chains observed in its parental strain (A) or other S. salivarius strains. This unique property confers VAR6 the ability to increase its biomass growth and co-adhesion property for efficient production and accumulation of bioactive antimicrobial peptides.

FIG. 4(A) shows a schematic representation of the bacteriocin locus on the megaplasmid of VAR6. Arrows indicate the direction of transcription. The predicted promoter region immediately upstream of the predicted operons is indicated by P.

FIG. 4(B) shows a global amino acid alignment of Pep1 (SEQ ID NO.: 5), Pep2 (SEQ ID NO.: 6), Pep3 (SEQ ID NO.: 7) and Pep4 (SEQ ID NO.: 8) peptides using Clustal O (v.1.2.4). Gaps necessary to increase the sequence similarity are indicated by dashes. Asterisks indicate the conserved residues for all four peptides. Colons indicate the conservation between residues of strongly similar properties. Periods indicate conservation between residues of weakly similar properties. The sequence of predicted mature peptides is underlined.

FIG. 5A shows a schematic representation of the Pez-like plasmid maintenance system on the megaplasmid of VAR6. Arrow indicates the direction of transcription. The predicted promoter region immediately upstream of the predicted operon is indicated by a P.

FIG. 5B shows the deduced amino acid sequence of the PezA and PexT proteins. PezA contains a conserved helix-turn-helix domain (underlined) belonging to the XRE family of transcriptional regulator to regulate the transcription of the entire operon. The conserved zeta protein domain found in PezT that secures stable plasmid inheritance during cell division is shown in bold.

FIG. 6 shows the growth kinetics of LAB816 and VAR6 strains in a variety of media.

FIG. 7 shows the inhibitory activity of the VAR6 strain towards Micrococcus luteus (S795), S. pyogenes (S796), and S. mutons (S1) on two bacteriocin-producing agar media, in a deferred antagonism assay. BAC7 is composed of animal origin proteins while BAC8 is composed entirely of animal origin-free constituents.

FIG. 8 shows the inhibitory activity of the VAR6 strain towards clinical strains of S. mutons collected from children with severe early childhood caries, in a deferred antagonism assay. S. mutons S717 (serotype f), S. mutons S953 (serotype k), S. mutons S713 (serotype c), and S. mutons S818 (serotype e) cultivated on bacteriocin-producing agar media. BAC7 is composed of animal origin proteins while BAC8 is composed entirely of animal origin-free constituents.

FIG. 9 shows the killing of S. mutons biofilms by VAR6 as compared to chlorhexidine, BLIS K12® (K12), and BLIS M18® (M18). 24-h-old static biofilms of S. mutons S125 were developed in polystyrene microtiter plates using BHI medium. After 24 h of biofilm growth, the planktonic phase was removed and the biofilm layer was treated with chlorhexidine (CHX), BLIS K12® (KU), BLIS M18 (M18), or VAR6 in fresh BAC7 or BAC8 broth for 24 h at 37 degrees C. The biofilm-treated cells were detached, serially diluted, and spot-plated onto BHI-erythromycin agar plates to determine the percentage of biofilm cell survival from plate counts.

FIG. 10 shows the killing of S. mutans biofilms with VAR6 as compared to BLIS M18. 24-h-old static biofilms of S. mutans S125 were developed in polystyrene microtiter plates using BHI medium. After 24 h of biofilm growth, the planktonic phase was removed and the biofilm layer was treated with BLIS M18 (M18), or VAR6 in fresh BAC8 broth supplemented with high concentration of sucrose for 24 h at 37 degrees C. The biofilm-treated cells were detached, serially diluted, and spot-plated onto BHI-erythromycin agar plates to determine the percentage of biofilm cell survival from plate counts.

FIG. 11 shows the killing of S. mutans and C. albicans biofilms with VAR6. Static biofilms (24-h-old) of mono-species (S. mutans), dual-species (S. mutans-C. albicans) and dental plaque biofilms containing S. mutans were developed in a nutrient-rich medium. After 24-h of biofilm growth, the planktonic phase was removed, and the biofilm layer was treated with VAR6 in fresh BAC7 broth for 24 h at 37 degrees C. The biofilm-treated cells were detached, serially diluted, and spot-platted onto BHI-erythromycin agar plates to determine the percentage of biofilm cell survival from plate counts.

FIG. 12 shows cell adhesion to hydroxyapatite (HA) disc. A 24-h-old biofilm was formed on saliva-coated HA discs by BLIS K12® (A) and VAR6 (B) in the bacteriocin-producing medium BAC7. After 24 h of growth, the discs were washed in sterile phosphate-buffered saline (PBS, pH 7.2) to remove non-adherent cells. Cells were then dehydrated through ethanol rinses, critical point dried with liquid CO₂, mounted, and spatter coated with gold. Samples were examined using a scanning electron microscope at 500× (left) and 1000× (right).

FIG. 13 shows the stability of the antibacterial activity of Alg-VAR6-Ch encapsulated capsules against M. luteus over a 28-day period. Alg-VAR6-Ch microcapsules were prepared and stored at 4 degrees C. and assayed at days 0, 7, 14, 20 and 28. Note the relative stability of the inhibitory activities across the 28 days, with the inhibitory zones showing a range from 3.1±0.79 to 3.8±0.26 mm.

FIG. 14 shows cell survival of Alg-VAR6-Ch at days 0, 1, 2, and 5. The number of VAR6 dropped slightly within 2 days; by 5 days, the number of cells that survived dropped to less than 30%.

FIG. 15 shows cell survival (A) and inhibitory activities (B) of Alg-VAR6-Ch incubated at 4, 22, 37 and 68 degrees C. for 2 h. Note the total absence of cell viability and inhibitory function of the Alg-VAR6-Ch microcapsules at 68° C.

### DESCRIPTION

LAB813, a previously characterized S. salivarius, incorporated herein by reference, and available as Genbank accession numbers CP040803 [megaplasmid pSAL813] and CP040804 [chromosome] was first isolated from the dental plaque of a healthy non-carious child in a screen for S. salivarius strains with anti-MS activities. This strain of S. salivarius was used as a starting point to selectively grow and isolate a spontaneous variant of LAB813, which has been named VAR6 and which possesses improved antibacterial activity against a variety of caries pathogens, including S. mutans, and is able to grow on common dietary sugars in the presence of 2-deoxyglucose.

S. salivarius VAR6 characterization includes that it expresses a novel multipeptide lantibiotic bacteriocin which we have named SalE4. SalE4 is comprised of four peptides which are encoded on a megaplasmid. The genome of VAR6 also contains two systems (R/M, and an addiction system) for DNA protection and plasmid stabilization, which provides VAR6 with the advantageous properties of plasmid stability. VAR6 was found to possess potent antimicrobial activity towards S. mutans, an important agent implicated in the formation of dental caries. In comparative studies, VAR6 exhibited significantly improved S. mutans and other microbial killing/inhibitory activity as compared to commercially available S. salivarius strains BLIS M18® and BLIS K12®.

S. salivarius VAR6 was also found to be a K-minus strain of S. salivarius and was found to express cell surface appendages for adhesion in the oral cavity. It provided better adhesion to saliva-coated hydroxyapatite discs, which were used as a proxy for a tooth surface, indicating it may have a longer acting effect. VAR6 was found to possess potent anti-biofilm activity in both plant-based and animal-based protein media, was able to grow in the presence of all common dietary sugars, and well tolerated 2-deoxyglucose analog. VAR6 also had the advantageous property of being able to grow in the presence of xylitol (a sugar substitute found in many chewing gums) and showed enhanced antimicrobial activity towards S. mutans in the presence of xylitol.

Thus, S. salivarius and its expressed multipeptide lantibiotic bacteriocin SalE4, are useful as an oral probiotic for the prevention of dental caries. Since biofilm formation plays an important role at the junction of the tooth and gum, S. salivarius and SalE4 are likely also useful in the treatment or prevention of periodontal diseases.

As used herein, the term “VAR6” is intended to apply to the newly isolated strain of S. salivarius described herein, as well as strains that are derived therefrom, for example, selectively isolated strains which utilize the S. salivarius strain described herein as a parent.

As used herein, the term “SalE4” is intended to apply to polypeptide lantibiotics produced by the S. salivarius strain VAR6, as well as biologically functional variants produced by other S. salivarius strains. Therefore, the term “SalE4” includes, for example, allelic variants that are produced by other strains of S. salivarius or other closely related strains of other species.

The invention includes biologically functional variants of a composition comprising Pep1 (SEQ ID NO:5), Pep2 (SEQ ID NO:6), Pep3 (SEQ ID NO:7) and Pep4 (SEQ ID NO:8), or having as pre-peptides these sequences, including those having about 90% to about 110% of the biological activity of a polypeptide comprising Pep1 (SEQ ID NO:5), Pep2 (SEQ ID NO:6), Pep3 (SEQ ID NO:7) and Pep4 (SEQ ID NO:8). Such variants can include amino acid substitutions selected according to general rules known in the art so as to have little effect on activity. Typically, the Pep1 (SEQ ID NO:5), Pep2 (SEQ ID NO:6), Pep3 (SEQ ID NO:7) and Pep4 (SEQ ID NO:8) form a single multipeptide lantibiotic where the four peptides are bound with thioether bridges. Each of Pep1, Pep2, Pep3, and Pep4 may undergo several post-translational modification events, including dehydration of specific hydroxyl amino acids and formation of thioester amino acids via addition of neighboring cysteines to didehydro-amino acids. Further post-translational processing involves cleavage of a leader sequence, which can be coincident with transport of the mature molecule to the extracellular space.

The genetics of lantibiotic production have been studied in several species of bacteria. In general, it has been found that a structural gene for a preprolantibiotic is clustered with genes that encode products responsible for post-translational modifications of the lantibiotic. In certain instances, these genes are known to form an operon or operon-like structure (see e.g., Schnell, et al. (1992) Eur. J. Biochem. 204:57-68). Production of lantibiotics can also require accessory proteins, including processing proteases, translocators of the ATP-binding cassette transporter family, regulatory proteins, and dedicated producer self-protection mechanisms. For example, at least seven genes have been shown to be involved in epidermin biosynthesis.

The discovery of new lantibiotic compounds having antibiotic activity can be particularly important in view of the increased resistance to presently available antibiotics in certain pathogenic microorganisms. Novel lantibiotic compounds having unique or superior activity against particularly virulent pathogenic bacteria are desirable in providing new weapons in the arsenal against bacterial infection, in particular, against oral bacteria such as the bacteria associated with the formation of caries.

Another embodiment of the invention comprises a pre-lantibiotic of SEQ ID NO:5, SEQ ID NO:6, SEQ ID NO:7, or SEQ ID NO:8, and variants thereof. A pre-lantibiotic is a form of a lantibiotic peptide that does not have thioether bridges formed.

Lantibiotic molecules of the present invention also include fusion of a lantibiotic polypeptide with another compound, such as a compound to increase the stability and/or solubility of the polypeptide (e.g., polyethylene glycol) and fusion of a polypeptide with additional amino acids, such as an IgG Fc fusion region peptide, a leader or secretory sequence, or a sequence facilitating purification.

Lantibiotics of the present invention can be isolated from culture medium in which its native host organism, i.e., VAR6, has been grown. In addition, other coding sequences important in post-translation modification can be cloned and transformed into a S. salivarius species or strains of other bacterial species such that a recombinant lantibiotic is produced. See, e.g., Qi et al., Appl. Environ. Microbiol. 66:3221-3229 (2000).

A potential complexity in the introduction of the phenotype for SalE4 into a new strain is the fact that the peptide undergoes post-translational modifications by other genetic elements in the host strain. These genetic elements should be present in a host strain or should be transferred to a host strain along with a polynucleotide encoding a lantibiotic of the invention in order to provide proper post-translational modification of a recombinant lantibiotic. Post-translational modification genes are contained within the genome of strain SalE4. Mutagenesis studies on SalE4 can identify all necessary components for post-translational modification. In another embodiment of the invention, lantibiotics can be synthesized ex vivo. A number of techniques exist for the synthesis of polypeptide molecules by relatively conventional organic chemical techniques. For example, solid phase polypeptide synthesis permits the creation of polypeptides the size of lantibiotics of the invention such that they can readily be synthesized outside of a microbial host.

A lantibiotic of the invention can be used as an antibiotic. Since a lantibiotic is produced by a common S. salivarius strain present in human mouths, it is expected to be relatively non-toxic to human species and other animals. This conclusion is further buttressed by its analogous characteristic to existing S. salivarius antibiotics, such as BLIS KU, which are known to be quite non-toxic to mammals. While primarily for use to prevent oral bacteria-caused diseases and conditions, such as caries, lantibiotics of the invention can be applied to an area in which it is desired to inhibit microbial growth. For example, a lantibiotic of the invention can be used in a dentifrice. A dentifrice is a composition used for cleansing teeth. A dentifrice can also comprise, for example, abrasives, detergents, binders, medicaments, caries preventatives, excipients, carriers, and flavoring agents. A dentifrice can be a liquid, paste or powder.

In one embodiment of the invention a lantibiotic is bacteriostatic, that is, it inhibits the growth or multiplication of bacteria. In another embodiment of the invention a lantibiotic is bacteriocidal, that is, it kills bacteria. A lantibiotic can have bacteriostatic action, bacteriocidal action, or both activities against a given bacterium.

Lantibiotics of the invention can be used to control bacterial growth. “Control bacterial growth” means to reduce bacterial growth, to reduce bacterial multiplication, to kill bacteria, or combinations thereof. For example, a lantibiotic can be applied to an area in which it is desired to control bacterial growth. An area can be, for example, animal tissue, such as human tissue, food, plants, or an inanimate object. Where a lantibiotic of the invention is used to control the growth of bacteria in an animal, a pharmaceutical composition comprising a lantibiotic of the invention is administered to the animal. The growth of the bacteria is controlled. The animal can be any type of mammal or non-mammal. In one embodiment of the invention, the animal is a human.

In one embodiment of the invention a lantibiotic is used to treat or ameliorate a bacterial infection in an animal or a human. The lantibiotic is administered to the animal/human and the infection is treated or ameliorated.

A therapeutically effective dose or effective amount of a lantibiotic of the invention refers to that amount of lantibiotic that controls bacterial growth in a desired location.

A therapeutically effective dose or effective amount can be estimated initially either in bioassays or in animal models, such as mice, rabbits, dogs, or pigs. An animal model can also be used to determine the appropriate concentration range and route of administration. Such information can then be used to determine useful doses and routes for administration in, for example, humans. Therapeutic efficacy and toxicity can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. In one embodiment of the invention, pharmaceutical compositions exhibit large therapeutic indices. The data obtained from bioassays and animal studies is used in formulating a range of dosage for animal use. The dosage contained in such compositions can be within a range of circulating concentrations that include the ED₅₀ with little or no toxicity. The dosage varies within this range depending upon the dosage form employed, sensitivity of the patient, and the route of administration.

An exact therapeutically effective dosage will be determined by a practitioner of skill in the art, in light of factors related to the animal that requires treatment. Dosage and administration are adjusted to provide sufficient levels of a lantibiotic or to maintain the desired effect. Factors that can be taken into account include the severity of the bacterial infection, general health of the subject, age, weight, and gender of the subject, diet, time and frequency of administration, drug combination(s), reaction sensitivities, and tolerance/response to therapy.

An effective amount of lantibiotic to be applied to an inanimate object or to food can be determined by one of skill in the art using routine experimentation.

Compositions of the invention can also contain excipients, such as water, saline, glycerol, sucrose, alcohols, invert sugar, glucose, polyols fats, waxes, semisolid and liquid polyols, natural or hardened oils, lactose, corn starch, talc, stearic acid, mixed polymers of glycolic acid and lactic acid dextrose, maltodextrin, ethanol, or the like, singly or in combination. Compositions of the invention can also comprise substances such as wetting agents, emulsifying agents, or pH buffering agents. Liposomes can also be used as a carrier for a composition of the invention, see e.g., N. Weiner, Drug Develop. Ind. Pharm. 15:1523 (1989); “Liposome Dermatics” (Springer Verlag 1992) and Hayashi, Gene Therapy 3:878(1996). Further details on techniques for formulation and administration can be found in the latest edition of Remington's Pharmaceutical Sciences (Maack Publishing Co., Easton, Pa.). Probiotic compositions of the invention may include lyophilized powders.

Pharmaceutical compositions for oral administration can include, for example, tablets, pills, dragees, capsules, liquids, gels, syrups, slurries, suspensions, and the like, for ingestion by the patient, or for rinsing in the mouth. Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can include substances that increase the viscosity of the suspension, such as sodium carboxymethyl cellulose, sorbitol, or dextran. Additionally, suspensions of a lantibiotic of the invention can be prepared as oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers can also be used for delivery. Optionally, the suspension can also contain suitable stabilizers or agents that increase the solubility of the compounds to allow for the preparation of highly concentrated solutions.

Lantibiotics of the present invention can be administered in an isolated protein form, or in a living or dead bacteria comprising the lantibiotic, for example, through the administration of live VAR6 culture as a probiotic, or of dead VAR6 culture containing the lantibiotic.

Probiotic formulations comprising VAR6 culture are an effective means for administering the compositions of the present invention. Probiotic compositions may include prebiotic ingredients, such as lactitol, inulin, or a combination thereof. The probiotic compositions may also include excipients or stabilizers, such as dicalcium phosphate, dextrose, fructose, stearic acid, citric acid, or other ingredients common in other commercial probiotic formulations. For example, we have shown an additive effect of xylitol in increasing inhibitory activities against S. mutans, and as such the probiotic compositions may include xylitol. The composition can be formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like. For administration to children, the product may be flavoured (e.g., fruit flavored, such as blueberry) and may be in a variety of shapes that are pleasing to children (stars, animals, popular characters, smiley faces, etc.). The product may be formulated as part of a gummy candy in order to improve acceptance by children.

A method of administration or use of the composition for improving oral health comprises oral administration of the composition by chewing twice daily for a sequential period of days sufficient to produce a measurable improvement in at least one oral health parameter.

It should be understood that the examples and embodiments described herein are for illustrative purposes only and that various modifications or changes in light thereof will be suggested to persons skilled in the art and are to be included within the spirit and purview of this application and the scope of the appended claims. All references cited in this disclosure are incorporated by reference in their entirety.

### EXAMPLE 1

### Isolation of Streptococcus salivarius VAR6

LAB813, a previously characterized S. salivarius strain (Gong S-G, Chan Y, Lévesque C M. Microbiol Resour Announc 2019 Oct. 10; 8 (41):e01092-19, incorporated herein by reference, and available as (Genbank accession numbers CP040803 [megaplasmid pSAL813] and CP040804 [chromosome]) was first isolated from the dental plaque of a healthy non-carious child in a screen for S. salivarius strains with anti-MS activities. S. salivarius LAB813 was cultivated on Mitis Salivarius Agar (Difco®) plate at 37° C. overnight. The preculture was used to inoculate (1% (v/v)) fresh broth of TYE supplemented with 11 mM lactose (control), 5.5 mM glucose or 5.5 mM fructose, and the cultures were incubated statically at 37° C. until the mid-exponential phase (OD₆₀₀˜0.6) was reached. Cells were then passaged on TYE agar plate supplemented with 11 mM lactose and a lethal concentration (0.5 mM) of 2-deoxyglucose (2-DG) at 37° C. for 24 h. For the isolation of variants, colonies able to grow in the presence of 2-DG were cultivated on TYE agar supplemented with 11 mM mannose or 11 mM lactose. Variants displaying normal growth under lactose condition and altered growth on mannose were selected for molecular typing and phenotypic analysis. These included VAR6.

All bacterial strains used are listed in Table 1.

TABLE 1
Strains used.

| | | Reference |
|---|---|---|
| Strain | Relevant properties^{a} | or source |
| S. salivarius | | |
| LAB813 | Clinical isolate from a caries-free subject; | Lab stock |
| | 2-DG^{s} | |
| K12 | BLIS K12 ® | ATCC |
| M18 | BLIS M18 ® | ATCC |
| VAR6 | Spontaneous variant, 2-DG^{r} | Novel |
| VAR17 | Spontaneous variant, 2-DG^{r} | Novel |
| VAR47 | Spontaneous variant, 2-DG^{r} | Novel |
| VAR135 | Spontaneous variant, 2-DG^{r} | Novel |
| VAR177 | Spontaneous variant, 2-DG^{r} | Novel |
| VAR182 | Spontaneous variant, 2-DG^{r} | Novel |
| Targets | | |
| UA159 | S. mutans; serotype c | Lab stock |
| S125 | S. mutans; serotype c; Ery^{r} | Lab stock |
| S713 | S. mutans from S-ECC; serotype c | Clinical |
| S717 | S. mutans from S-ECC; serotype f | Clinical |
| S818 | S. mutans from S-ECC; serotype e | Clinical |
| S953 | S. mutans from S-ECC; serotype k | Clinical |
| S795 | Micrococcus luteus | Lab stock |
| S796 | Streptococcus pyogenes | Lab stock |

^{a}2-DG^{s}, 2-deoxyglucose sensitive; 2-DG^{r}, 2-deoxyglucose resistance; Ery^{r}, erythromycin resistance; S-ECC, severe-early childhood caries.

### Molecular Typing

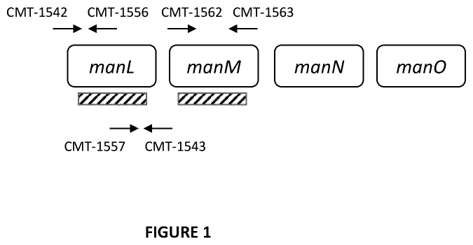

The parental strain and its variants were cultivated in a 10-m1 volume of TYE-lactose broth at 37° C. for 18 h without agitation. Total genomic DNA was extracted and treated with RNase A. PCR primers were specifically designed to amplify the 5′-end and 3′-end of manL gene of S. salivarius (FIG. 1). The primer pair CMT-1542 (5′-ATC TTT TCC GAC TCA TTG TC-3′ (SEQ ID NO.: 1)) and CMT-1556 (5′GAT TTT CTC CAG CAA TTC GG-3′ (SEQ ID NO.: 2) was used to amplify the 5′-end of manL (expected PCR product of 550 bp). The primer pair CMT-1557 (5′-CCG AAT TGC TGG AGA AAA TC-3′ (SEQ ID NO.: 3)) and CMT-1543 (5′-CGA ATG AAG GTT ATT GAA CG-3′ (SEQ ID NO.: 4)) was used to amplify the 3′-end of manL (expected PCR product of 770 bp). PCRs were carried out in 20-μl volumes using a 3-step profile: 1 min of denaturation at 94° C., 1 min of annealing at 56° C., and 1 min of extension at 72° C. for a total of 35 cycles. Variants were also screened by Southern blotting using an S. salivarius manL-specific Dig-labeled DNA probe amplified using the primer pair CMT-1542/CMT-1543 and an S. salivarius manM-specific Dig-labeled DNA probe amplified using the primer pair CMT-1562 (5′-CTC TCA GCT AAT CAC AAC TC-3′ (SEQ ID NO: 13) and CMT-1563 (5′-GTA GTC TTC CAA GAT ATC GC-3′ (SEQ ID NO: 14)). Standard protocols were used for probe labeling using Dig High Prime DNA Labeling kit (Sigma), gel electrophoresis, and Southern blotting.

### Growth Kinetics Analysis

The parental strain LAB813 and its variant VAR6 were cultivated statically in TYE broth supplemented with 0.2% (v/v) lactose and 0.5 mM 2-DG (for variants) for 16 h at 37° C. A 100-fold dilution was made into 300 μl of TYE supplemented with 0.2% (v/v) sugar and added to the individual wells of a 100-well Bioscreen C plate in triplicate. Growth in the presence of sub-MIC of xylitol was also tested in TYE-lactose supplemented with 0.5% (v/v) xylitol. Wells without cells were used as blank controls. A microbiology workstation (Bioscreen C, Growth Curves, USA) was employed to continuously grow cells and measure cell growth for 16 h at 37° C. The reader was equipped with Biolink software that allow automatic recording and conversion of optical density (OD) readings into growth curves. Measurements were recorded every 20 min with shaking to prevent cell aggregation.

### Bacteriocin Assays

A deferred antagonism assay was used to demonstrate antimicrobial activity. S. salivarius VAR6 was inoculated as a 1-cm wide streak across the surface of a bacteriocin-producing agar medium and incubated at 37° C. for 24 h. The visible growth of VAR6 was removed using a sterile cotton swab, and the surface of the agar was sterilized by exposure to chloroform vapours for 30 min. The plates were then exposed to air for 15 min before inoculation with overnight cultures of the target strains across the line of the original VAR6 strain growth. The plates were then incubated as before for 24 h and examined for zones of growth interference. Areas of inhibition of target strain growth were measured by the length of inhibition. All tests were performed in duplicate, and further examination was conducted if marked discrepancies were observed among the inhibition patterns observed.

### In Vitro Model for Growing Biofilms

Static biofilms of S. mutons were developed on polystyrene microtiter plates by inoculating 300 μl of BHI medium with 10 μl (10⁶ CFU/ml) of S. mutons S125 from an overnight culture. After 24 h of growth, the planktonic phase was carefully removed, and fresh bacteriocin-producing medium (BAC7, BAC8) containing chlorhexidine (10×MIC), BLIS M18® (10⁷ CFU/ml), BLIS K12® (10⁷ CFU/ml) or VAR6 (10⁷ CFU/ml) was overlayed onto established biofilms and the plates were incubated for 24 h at 37° C. The upper phase was then removed, and the biofilms were washed with sterile phosphate-buffered saline (PBS, pH 7.2) and dislodged by gentle scraping using sterile pipette tips. Biofilm cells were serially diluted and plated on BHI-erythromycin agar plates. Colonies of S. mutons were counted after 48 h of incubation at 37° C. in air with 5% CO₂. All assays were performed in triplicate from three independent cultures. For the fungal-bacterial assay, biofilms were developed in Yeast extract-Peptone-Glycerol (YPG) medium and using a mixture of S. mutons/Candida albicans at a ratio of 1:1. For the dental plaque assay, biofilms were developed in BHI broth and using BHI plaque suspension supplemented with S. mutons (approx. 10⁵ CFU/ml).

### Scanning Electron Microscopy

Biofilms of S. salivarius were developed on sterile saliva-coated hydroxyapatite (HA) discs (Clarkson Chromatography) in sterile 24-well polystyrene plates. Static biofilms were developed on HA discs by inoculating 1 ml of BAC7 medium with 30 μl (10⁷ CFU/ml) of VAR6 or BLIS K12® from an overnight culture. After 24 h of growth, the discs were washed in sterile PBS to remove the non-adherent cells. Cells were then dehydrated through ethanol rinses, critical point dried with liquid CO₂, mounted, and sputter coated with gold. Samples were examined using a scanning electron microscope (model Hitachi FlexSEM 1000). The biofilm morphology was evaluated at randomly selected points on each HA disc.

### Generation of Natural Variants of S. salivarius

PTS:glucose/mannose was targeted to develop spontaneous variants of the parental strain LAB813 with altered sensitivity to the toxic 2-deoxyglucose (2-DG) analog and an altered growth profile on mannose. These variants are usually defective in some components of the PTS:glucose/mannose system and contain modifications affecting either the PTS activity or the synthesis of its components. We designed a protocol using lactose, a sugar found in milk and dairy products, to select for variants resistant to the toxic glucose analog. In S. salivarius, lactose is a non-PTS sugar that is transported by a lactose permease and the enzymes involved in its metabolism are inducible. We thus cultivated the parental strain under glucose or fructose condition for 10-12 generations before selecting the variants on nutrient-rich agar supplemented with lactose and a lethal concentration of 2-DG analog. Because LAB813 can take up the glucose analog using its intact PTS:glucose/mannose system, the cells cannot grow on lactose in the presence of 2-DG since the lactose metabolism in S. salivarius is subject to catabolite repression. In contrast, variants of the parental strain that are unable to transport the glucose analog can easily grow since the metabolism of lactose is not repressed due to a genetic rearrangement of the PTS:glucose/mannose operon. These variants can thus be distinguished according to the resistance to the toxic analog and modified growth properties.

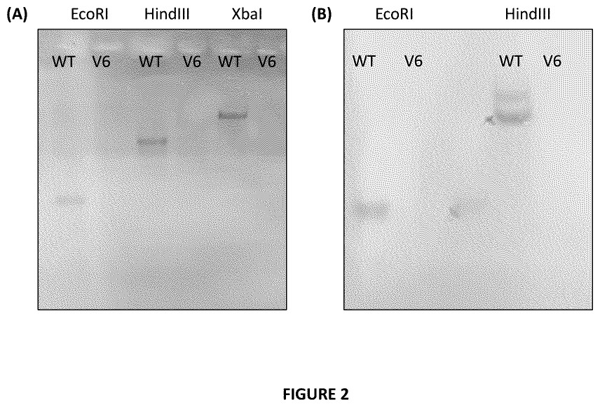

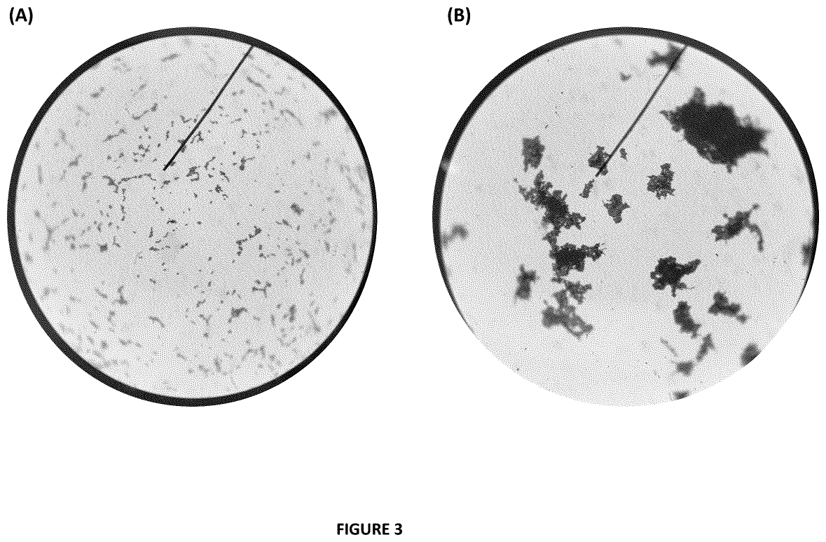

Six spontaneous variants of the parental strain were generated. VAR6, VAR17, VAR47, VAR135, VAR177, and VAR182 were isolated based on their resistance to the toxic glucose analog and impaired growth in the presence of mannose as the sole source of carbohydrate. Molecular analysis (PCR, hybridization) of the manLMNO locus encoding the PTS:glucose/mannose of S. salivarius revealed chromosomal rearrangement of the PTS:glucose/mannose multienzymatic system in variant VAR6 resulting in the deletion of manL gene encoding the enzyme IIAB (FIG. 2) (ManL peptide: SEQ ID NO: 11; ManM peptide: SEQ ID NO: 12). We next examined the variants by light microscopy following Gram staining since previous studies showed that ManL-deficient strains could exhibit alterations in the cellular envelope. We found that greater than 90% of VAR6 cells were not present as individual cocci or small chains but formed microcolonies and large cellular aggregates (FIG. 3). Advantages of cellular aggregates are higher bacterial fitness during competition, enhanced resilience towards stressful environmental conditions, and higher biomass for production of antimicrobial molecules.

Altogether, these results confirmed that VAR6 was a genetically and phenotypically distinct variant and was selected for further analysis.

### VAR6 Produces a Novel Antibiotic Bacteriocin

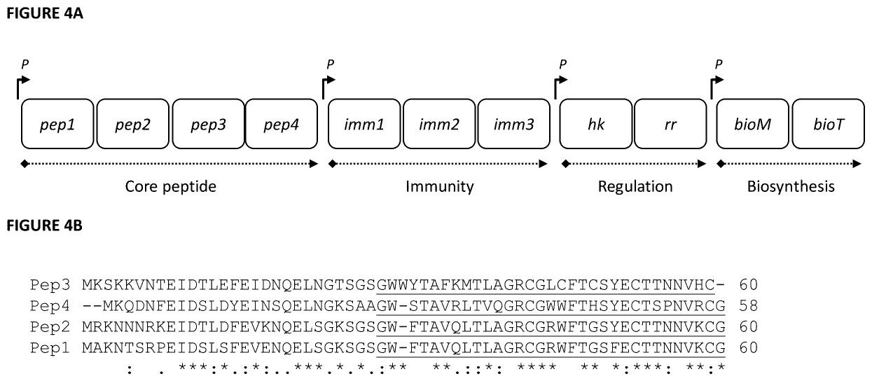

S. salivarius VAR6 contains a megaplasmid of 183,700 bp. The nucleotide sequence was analyzed to predict putative bacteriocin gene clusters and biosynthetic genes using BAGEL4 and antiSMASH Web servers. These pipelines predicted several putative bacteriocin genes located in a region of 11,594 bp. The bacteriocin gene cluster is organized into four predicted operon structures (core peptide, immunity, regulation, biosynthesis), all of which contain putative promoter region in the upstream region of the predicted operon (FIG. 4). All ORFS were unidirectionally oriented and were preceded by a conserved streptococcal ribosome-binding site suggesting efficient protein translation. The proteins encoded by the bacteriocin locus are presented in Table 2.

TABLE 2
Summary of the characteristics of the bacteriocin locus in VAR6

| | Size | | |
|---|---|---|---|
| ORF | (aa) | Homology^{a} | Putative function |
| Pep1 | 60 | ALO23662, SrnA′ salivaricin E precursor | Lantibiotic peptide |
| Pep2 | 60 | ALO23661, SrnA salivaricin E precursor | Lantibiotic peptide |
| Pep3 | 60 | AMB48460, PdlA3 lantibiotic protein | Lantibiotic peptide |
| Pep4 | 58 | WP_149561016, salivaricin M precursor | Lantibiotic peptide |
| Imm1 | 79 | ALO23664, ABC transporter ATPase | ABC transporter |
| Imm2 | 145 | VOB59524, ABC transporter ATPase | ABC transporter |
| Imm3 | 671 | QEM31565, ABC transporter permease | ABC transporter |
| HK | 524 | VIZ63382, histidine kinase | Two-component system |
| RR | 198 | ALO23670, response regulator | Two-component system |
| BioM | 984 | QEM31562, lantibiotic synthesis protein | Lantibiotic biosynthesis |
| BioT | 707 | ALO23672, ABC-type bacteriocin/lantibiotic | ABC transporter |
| | | exporters | |

^{a}Best BlastP hit in NCBI GenBank.

The core peptide operon encodes a novel array of four propeptides with amino acid sequences similar to those of salivaricin M and salivaricin E in S. salivarius and shown as Pep1 (SEQ ID NO:5), Pep2 (SEQ ID NO:6), Pep3 (SEQ ID NO:7) and Pep4 (SEQ ID NO:8) in FIG. 3.

The core peptide operon encodes a novel array of four propeptides with amino acid sequences very similar to those of salivaricin M and salivaricin E in S. salivarius. Gene expression analysis confirmed that the four peptide genes were expressed under different sugar conditions tested suggesting that all four peptides play a role in the production of the bioactive bacteriocin.

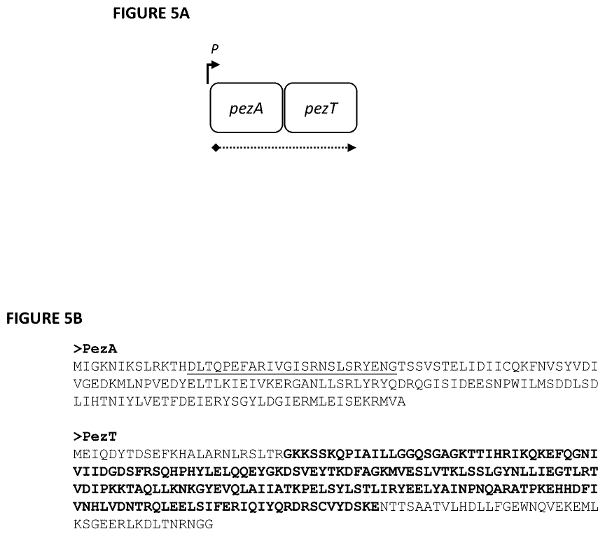

Attempts were made at inactivating the individual peptide-encoding genes using PCR ligation mutagenesis or a suicide vector. Despite our best efforts, we were unable to inactivate any genes encoding the lantibiotic bacteriocin. The presence of a Pez-like addiction system (FIG. 5) on the megaplasmid may ensure the maintenance of the lantibiotic locus. It is noteworthy that plasmid curing experiments using different curing agents (e.g., novobiocin, acriflavine) were also all unsuccessful. The Pez-like plasmid maintenance system may be guarding against DNA loss in VAR6 and secures stable inheritance of the plasmid during bacterial cell division. Since a functional lantibiotic bacteriocin locus is needed to provide the antimicrobial activity by a probiotic candidate, the presence of the Pez-like system on the megaplasmid of VAR6 thus represents an advantage over the commercially available strains BLIS K12 and BLIS M18.

### VAR6 Can Grow on Common Dietary Sugars

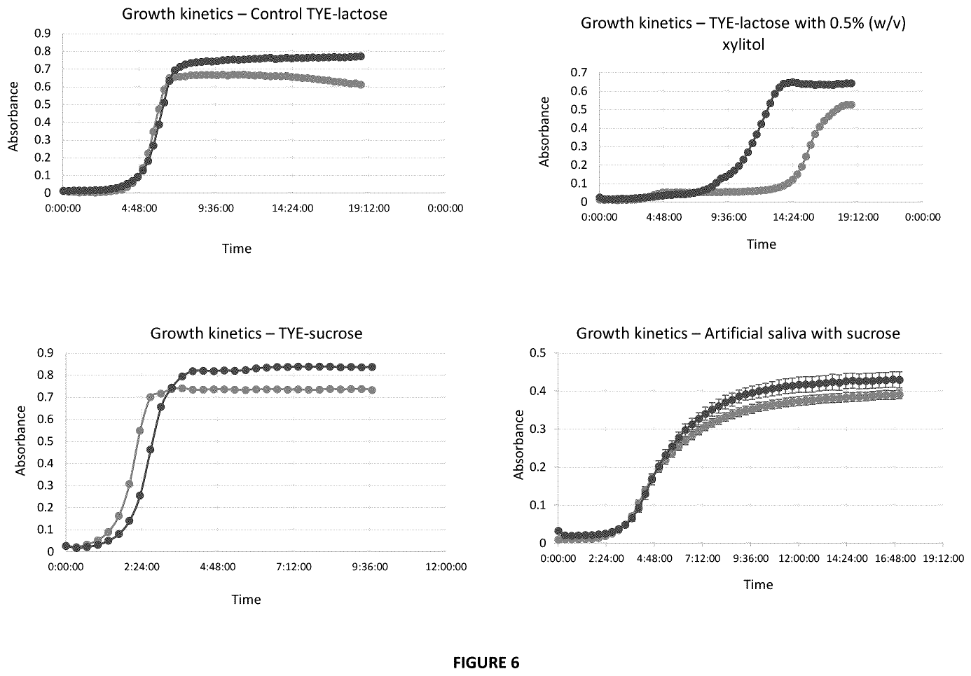

We found that VAR6 could be easily cultivated in several animal origin peptone-based media used for microbial fermentation and in animal-free enriched complete media as used in the manufacture of biopharmaceuticals for the human health market. Results obtained using different host diet sugars showed that VAR6 could be cultivated in nutrient-rich broth supplemented with sucrose (table sugar) or lactose (milk sugar), two of the most common sugars encountered in American diets (FIG. 6). When compared to the parental strain LAB813, VAR6 showed a reduced lag phase and produced higher growth yield (˜25% increase) under the same growth conditions, suggesting a better adaptation of VAR6 when introduced into a rich environment. VAR6 could also grow at higher biomass (˜10-25% increase) in artificial saliva (saliva substitute) and in the presence of xylitol, a sugar substitute found in chewing gum and used as a preventive measure as a cariostatic agent. Since the volumetric bacteriocin production is dependent on the total biomass production, VAR6 strain appears to be better suited for optimal biomass accumulation and bacteriocin production that the parental strain LAB813.

### VAR6 Possesses Potent Anti-S. mutans Activity

S. salivarius VAR6 was easily cultivated and maintained under standard growth conditions and produced abundant antimicrobial peptides when grown in growth media of both animal and vegetal origin. It was found that some growth media provided improved production of antimicrobial peptides. Several bacteriocin-production media were prepared and tested for their ability to enhance bacteriocin production. Table 3 lists such BAC media.

TABLE 3
BAC media developed for microbial fermentation.

| | | | Growth | |
|---|---|---|---|---|
| Medium | Components | Sugar | yield^{a} | Efficacy^{b} |
| BAC1 | Animal origin | Glucose | Poor | No |
| BAC2 | Animal origin | Glucose | Good | No |
| BAC3 | Animal origin | Glucose | Good | No |
| BAC4 | Animal origin | Glucose | Good | No |
| BAC5 | Animal origin | Glucose | Poor | No |
| BAC6 | Animal origin | Glucose | Good | (+/−) |
| BAC7 | Animal origin | Sucrose | Good | Yes |
| BAC8 | Vegetal origin | Sucrose | Good | Yes |

^{a}Biomass production obtained after 24 h of incubation.
^{b}Efficacy against clinical isolates of S. mutans in a deferred antagonism assay.
Yes: inhibitory activity observed; No: inhibitory activity not observed; (+/−): weak activity observed

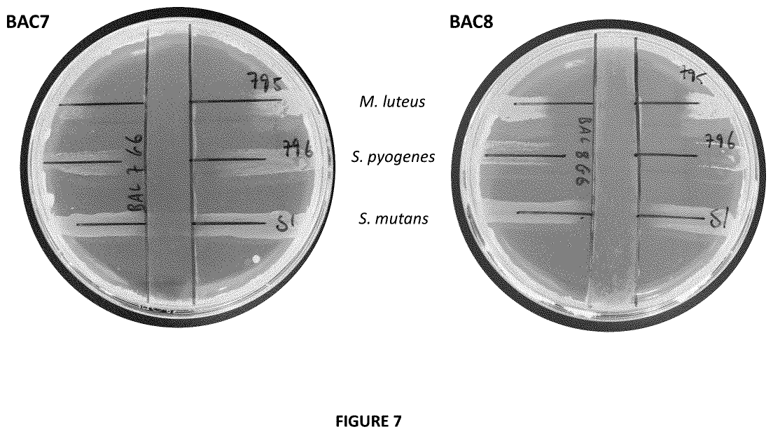

A deferred antagonism assay was used to detect antibacterial activity against three standard targets: Micrococcus luteus, Streptococcus pyogenes, and S. mutons. Antimicrobial activity was detected on BAC6, BAC7, and BAC8. BAC7 showed the highest performance against S. mutons. BAC7 medium was composed of protein hydrolysates derived from the partial hydrolysis of proteins from animal sources. BAC8 medium was designed to provide the same high level of performance by replacing the animal origin proteins of BAC7 with water-soluble products derived from the partial hydrolysis of proteins from plant and yeast sources. Results obtained using BAC8 production medium showed that VAR6 is capable of potent antibacterial activity in an enriched complete medium formulation consisting entirely of animal origin-free components. The results presented at FIG. 7 showed that VAR6 cultivated on BAC7 and BAC8 conserved its antimicrobial activity towards all tested target laboratory strains. VAR6 was further tested against a panel of clinical isolates of S. mutons collected from children with severe-early childhood caries (Table 4).

TABLE 4
S. mutans clinical isolates tested in this study.

| Isolate | Characteristic | Origin |
|---|---|---|
| S713 | Serotype c; caries-active subject | Canada |
| S717 | Serotype f; caries-active subject | Canada |
| S724 | Serotype c; caries-active subject | Canada |
| S727 | Serotype c; caries-active subject | Canada |
| S731 | Serotype c; caries-free subject | Canada |
| S736 | Serotype c; caries-active subject | Canada |
| S754 | Serotype c; caries-free subject | Canada |
| S757 | Serotype c; caries-active subject | Canada |
| S764 | Serotype c; caries-active subject | Canada |
| S768 | Serotype c; caries-active subject | Canada |
| S769 | Serotype c; caries-active subject | Canada |
| S779 | Serotype c; caries-active subject | Canada |
| S780 | Serotype c; caries-active subject | Canada |
| S781 | Serotype c; caries-active subject | Canada |
| S804 | Serotype e; caries-free subject | Canada |
| S810 | Serotype c; caries-free subject | Canada |
| S816 | Serotype e; caries-free subject | Canada |
| S817 | Serotype e; caries-free subject | Canada |
| S818 | Serotype e; caries-free subject | Canada |
| S819 | Serotype c; caries-free subject | Canada |
| S822 | Serotype c; caries-free subject | Canada |
| NN2117 | Serotype f | Japan^{a} |
| NN2141 | Serotype f | Japan^{a} |
| NN2165 | Serotype f | Japan^{a} |
| NN2193-1 | Serotype k | Japan^{a} |
| NN2323M-1 | Serotype k | Japan^{a} |
| TLJ11-2 | Serotype k | Japan^{a} |

^{a}Isolates kindly obtained from Prof. K. Nakano, Osaka University

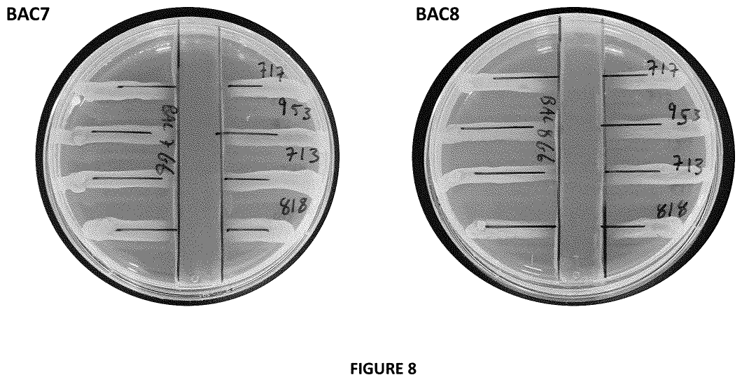

Through the deferred antagonism assay, an inhibition of all S. mutons strains was observed, including even the strains of the highly invasive serotype f. A representative figure showing the inhibitory activity of VAR6 against four selected target MS strains on BAC7 and BAC8 was shown in FIG. 8. The anti-S. mutons activity was destroyed by proteinase K treatment but not by heat treatment suggesting that the novel multipeptide lantibiotic produced by VAR6 was of proteinaceous nature and possessed heat stability.

### VAR6 Possesses Potent Anti-GAS Activity

Some strains of S. salivarius display antimicrobial activity against R-hemolytic group A Streptococcus (GAS or Streptococcus pyogenes). GAS is a ubiquitous pathogen causing infections of the upper respiratory tract (pharyngitis) or skin (impetigo). Infection with specific serotypes (M types) can lead to life-threatening diseases such as necrotizing fasciitis (‘flesh-eating’ disease) and streptococcal toxic shock syndrome. We thus screened VAR6 against GAS stocked in our bacterial collection (Table 5). When tested using a deferred antagonism assay, VAR6 was able to inhibit the growth of all GAS tested, even the M types (M28 and M4) frequently associated with invasive disease.

TABLE 5
Group A Streptococcus (GAS) isolates tested in this study.

| Isolate | M type | Clinical presentation |
|---|---|---|
| S796 | M52 | Impetigo |
| S799 | M4 | Pharyngitis; acute glomerulonephritis |
| S801 | M28 | Necrotizing fasciitis |
| S802 | M87 | Pharyngitis |
| S876 | M49 | Pyoderma-associated nephritis |

### VAR6 Possesses Strong Antimicrobial and Anti-Biofilm Properties

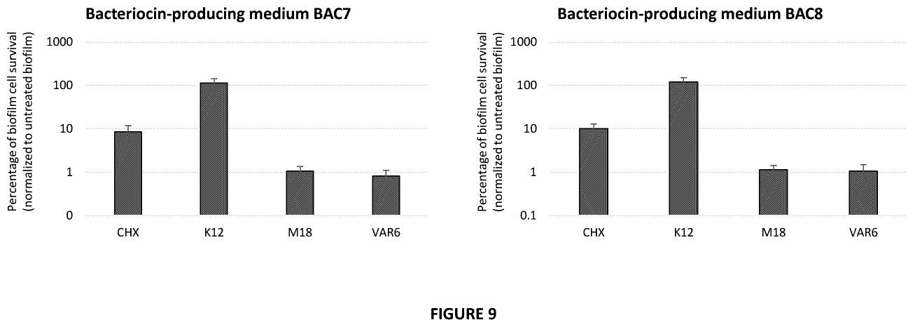

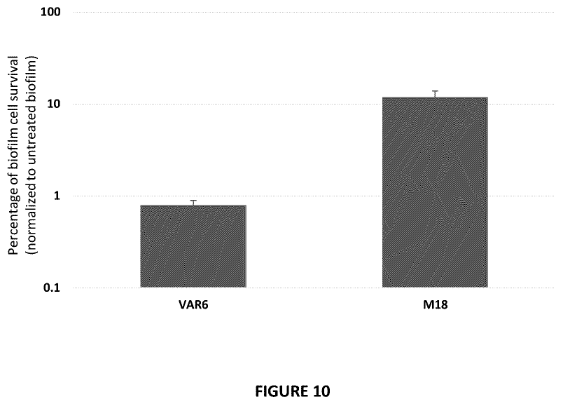

The inhibitory activity of VAR6 against S. mutons biofilms was determined using a static microtiter plate assay, a standard method in biofilm studies. Results showed that VAR6 significantly killed 99% (99.2%±0.29 in BAC7; 99.0%±0.43 in BAC8) of biofilm cells compared to ≈90% (91.6%±3.29 in BAC7; 90.1%±2.89 in BAC8) for chlorhexidine at 10X MIC, a broad-spectrum antimicrobial agent used routinely in dentistry (FIG. 9). In all experiments, the activities of the commercially available S. salivarius strains, BLIS K12® and BLIS M18® were used to compare against data obtained with VAR6. As expected, BLIS K12® was not capable of inhibiting the growth of the S. mutons biofilm cells. Although BLIS M18® could inhibit the growth of S. mutons biofilm cells, it was less efficient than VAR6 (M18: 88.2%±2.0 vs. VAR6: 99.2%±0.1) in a medium consisting entirely of animal origin-free components supplemented with concentration of sucrose (FIG. 10).

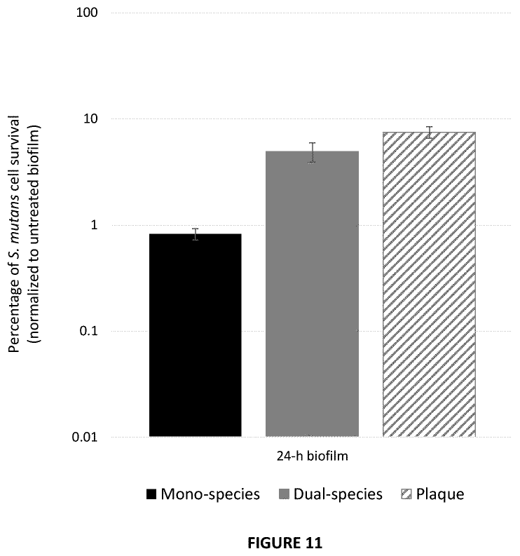

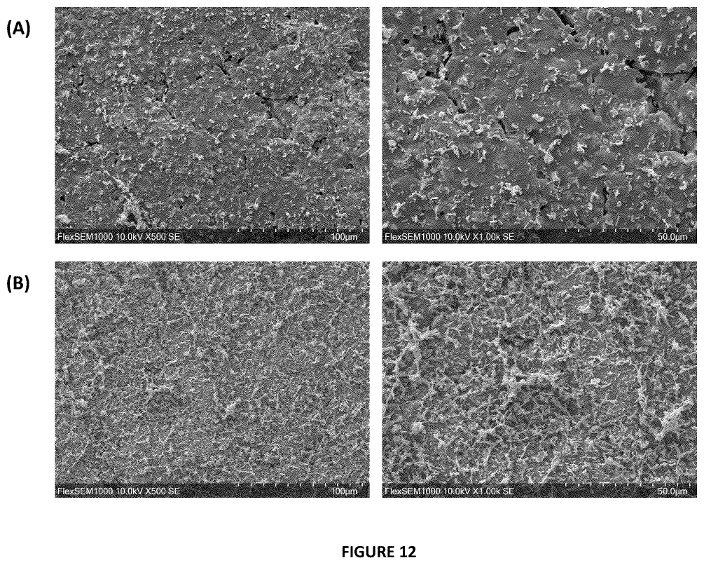

Because cariogenic biofilms are composed of mixed flora in vivo, we tested the inhibitory activity of VAR6 towards S. mutons in more complex biofilm structures. When tested in a fungal-bacterial biofilm composed of S. mutons and C. albicans, addition of VAR6 killed ≈95% (95.1%±1.0) of S. mutons cells (FIG. 11). VAR6 also showed effectiveness at inhibiting S. mutons in a multi-species plaque biofilm with cell killing ≈90% (92.5%±1.32).

### VAR6 Possesses Strong Adhesion Potential

VAR6 was found to encode proteinaceous hair-like appendages called fimbriae. Because fimbriae also play an important role in colonization, we tested the ability of VAR6 to attach to saliva-coated hydroxyapatite disks to mimic the natural tooth surfaces. BLIS K12® was used to compare against data obtained with VAR6 since it has been previously shown that K12 strain was capable of auto-aggregation and host-cell interactions. Our results showed that VAR6 cultivated in the presence of sucrose or lactose formed dense confluent films suggesting that the fimbriae may be acting as adhesin for the binding of the cells to the oral surfaces. We observed the films using scanning electron microscopy at 500× and 1000× and discovered that VAR6 had higher adhesion efficiency compared to K12 strain (FIG. 11).

### Protein Extracts From VAR6 Provide S. mutans Biofilm Inhibiting Activity

VAR6 is grown in growth media and peptides are extracted and isolated. The proteins comprise a lantibiotic bacteriocin multipeptide (which we named SalE4) composed of 4 peptides (which we named Pep1, Pep2, Pep3, and Pep4) having the sequences of SEQ ID Nos.: 5, 6, 7 and 8, respectively, and shown in FIG. 4B. Both living VAR6 and dead VAR6 isolates, as well as the isolated multipeptide, are found to kill S. mutans, and inhibit the growth of S. mutans biofilms, both in BAC7 and BAC8 medium, and in situ in the mouth of an individual.

### Recombinant Protein Extracts

Peptides Pep1, Pep2, Pep3 and Pep4 can be manufactured in known bacteria expression systems, by PCR amplifying the DNA sequence or inserting a DNA sequence which translates into the amino acid sequences of SEQ ID NO.: 5, 6, 7, or 8 (respectively) into a DNA vector containing a promoter region for expressing said sequence resulting into a DNA construct. The DNA vector may be linear or circular. The circular DNA vector may be a plasmid. The PCR amplified DNA or DNA vector can then be inserted in bacteria and the bacteria are grown in conditions that trigger the expression of the sequence. The peptides can be isolated from the grown bacteria, or from a supernatant of the bacterial growth medium. The lantibiotic bacteriocin multipeptide SalE4 can likewise be recombinantly produced, either by manufacturing Pep1, Pep2, Pep3 and Pep4 in different bacteria as described above, or by inserting four DNA sequences, each translating into one of the amino acid sequences of SEQ ID NO.: 5, 6, 7 and 8, into its own plasmid but inserting all four DNA constructs into a single bacterium or by creating a single DNA construct designed to express multiple peptides. Each of these techniques is well known in the art and, once the Pep1, Pep2, Pep3 and Pep3 peptides are expressed at sufficient concentration, they will spontaneously form the lantibiotic bacteriocin multipeptide SalE4.

### Oral Probiotic/Protein-Containing Products Comprising VAR6 and/or SalE4

Probiotic/protein-containing products can be made comprising VAR6 and/or SalE4, for use in the prevention of caries, for example, of caries caused by S. mutans, as well as for use in killing other cariogenic bacteria, including S. mutans, and inhibiting growth of cariogenic biofilms. For example, VAR6 can be grown and added, as a live bacterial culture, to a probiotic supplement to be used as a mouth rinse, a mouth wash, a toothpaste, in a candy, such as a gummy candy, in a drink, in a lozenge, or lollipop, using known methods. Alternatively, or in addition, isolated SalE4, either recombinantly produced or isolated from a grown VAR6 culture, can be similarly added. Such products can provide prevention of caries, inhibition of the growth of cariogenic bacteria, as well as inhibition of growth of biofilm and/or plaque in the mouth of an individual.

### Microencapsulated Product Comprising VAR6 and/or SalE4

For a probiotic to exert beneficial effects on the host, it is desirable that live cells of adequate dosage are able to reach the target site and survive and function. Microencapsulation was utilized to package the probiotics in protective shells, providing physical barriers and improving the viability and bioavailability of the probiotics.

Natural polymers are known as delivery carriers for probiotic microencapsulation, with alginate (Alg) being one of the most popular (Wang et al., Pharmaceuticals (Basel) 2022, 15(5) DOI: 10.3390/ph15050644). Alg is a linear polysaccharide consisting of β-(1-4)-linked D-mannuronic acid and α-(1-4)-linked L-glutaronic acid residues (Bonani W et al., Adv Exp Med Biol, 2020, 1250, 49-61. DOI: 10.1007/978-981-15-3262-7_4)Due to their derivation from brown algae, Alg exist in abundance in nature and are generally recognized as a safe and non-toxic material for bacterial encapsulation. When Alg interacts with an ionic cross-linking agent such as calcium chloride, a hydrogel matrix is formed that is permeable, making it easy for air and nutrient exchange, and survival of the probiotics, making the Alg hydrogel an excellent and popular carrier for delivering probiotics (Zhang et al., ACS Applied Materials & Interfaces, 2016, 8(14), 8939-8946. DOI: 10.1021/acsami.6b00191).

One challenge associated with Alg hydrogels is high porosity, which can be improved by combining with other materials such as chitosan (Ch) (Razavi, S et al., Food Hydrocolloids, 2021, 120, 106882). Ch is a non-toxic, biocompatible and biodegradable cationic polysaccharide with partly acetylated (1-4)-2-amino-2-deoxy-b-d glucan obtained from chitin. It is commonly used as a coating material for Alg beads and has been shown to increase the survivability of probiotics as compared to uncoated Alg beads (Muzzarelli, R. A. A. et al., Carbohydrate Polymers, 2012, 87 (2), 995-1012. DOI: https://doi.org/10.1016/j.carbpol.2011.09.063; Chávarri, M. et al., Int J Food Microbiol, 2010, 142 (1-2), 185-189. DOI: 10.1016/j.ijfoodmicro.2010.06.022; Cook, M. T. et al., Biomacromolecules, 2011, 12 (7), 2834-2840. DOI: 10.1021/bm200576h). The mechanism involves a negatively charged Alg that, upon interacting with positively charged Ch, generates a smoother surface with smaller porosity and less permeability to water-soluble molecules (Koo, S.; et al., Journal of Microbiology and Biotechnology, 2001, 11, 376-383; Krasaekoopt, W. et al., International Dairy Journal, 2004, 14 (8), 737-743. DOI: https://doi.org/10.1016/j.idairyj.2004.01.004).

A stock solution of 3.0% (w/v) Alg was made by mixing 3 g of sodium alginate (Sigma-Aldrich, Oakville, Canada) in 100 ml of de-ionized water and autoclaved. A stock solution of 1.5% (w/v) chitosan was made by dissolving 1.5 g of chitosan (medium molecular weight, Sigma-Aldrich, Oakville, Canada) in 100 ml 1% (v/v) glacial acetic acid solution. The pH was adjusted to 5.3 by adding 0.1 M NaOKH after which the solution was filtered and sterilized using UV light for 30 min.

Microbial strains were frozen at −80 degrees C. in nutrient rich liquid broth supplemented with 15% (v/v) glycerol. VAR6 was cultivated in BAC7 broth medium (10 g tryptone, 10 g yeast extract, 10 g peptone, 10 g neopeptone, 0.2 g NaCl, 0.25 g magnesium sulfate, 0.2 g sodium acetate, 0.5 g ascorbic acid, 2.5 g disodium phosphate per litre) supplemented with filtered-sterilized 0.5% (v/v) sucrose added after autoclaving the rest of the medium. Micrococcus luteus and Streptococcus pyogenes, indicator strains for the antimicrobial assays, were cultured in Brain Heart Infusion (BHI) broth. For the semisolid agar medium, the preparation was supplemented with 0.3% (w/v) agar.

A loop culture from VAR6 was inoculated in BAC7 broth containing 0.5% (v/v) sucrose and incubated overnight at 37° C. Batches of 1 ml of overnight grown VAR6 cells of approximately 10 9 CFU/ml were centrifuged at 12,000 rpm for 10 min and resuspended in 1 ml 1.5% (v/v) sodium alginate solutions. After mixing thoroughly, individual batches of the 1 ml Alg-VAR6 mix were released into a suspension bath of 1% (w/v) CaCl₂ solution to form capsules of ˜4-5 mm in diameter. After 30 min, CaCl₂ was removed, and the capsules were rinsed with PBS buffer and immersed in 0.1% (v/v) Ch (from a 1.5% stock solution) for 40 min. The Alg-VAR6-Ch thus formed were rinsed with PBS buffer and stored at 4° C. until further use.

The antimicrobial activity of the encapsulated VAR6 was studied via spot-on-lawn assays as described by Dufour et al. (Dufour, D. et al. Journal of Bacteriology, 2020, 202 (12), e00762-00719. DOI: doi:10.1128/JB.00762-19), and quantitated. Briefly, ˜10⁷ CFU/mL of M. luteus or S. pyogenes was overlayed on BHI agar plate. Next, wells of 9 mm diameter were created in the agar plates into which Alg-VAR6-Ch microcapsules and any controls were placed. The plates were incubated overnight at 37° C. and growth of inhibition was examined and quantitated. Quantitation of inhibitory activities was performed by averaging the zones of inhibition (ZOI) of each well.

Batches of Alg-VAR6-CH microcapsules were prepared as described above. Inhibitory activities of these experiments were assayed by spot-on-lawn as described above.

For the determination of the optimal VAR6 cell numbers to be incorporated in 1 ml of Alg (as described above), three concentrations were tested: 1×(1×10⁹ CFU/ml), 5×(5×10⁹ CFU/ml) and 10×(10×10⁹ CFU/ml) was used in every 1 ml of Alg.

For the stability assays, batches of microcapsules were prepared as described with one group assayed for inhibitory assays on Day 0, while the rest were kept at 4° C. and assayed for inhibitory activities at days 7, 14, 20, and 28.

For the temperature sensitivity experiments, batches of microcapsules were prepared and placed for 2 h at 4 different temperatures i.e., 4° C., 22° C., 37° C. and 68° C. In addition to spot-on-lawn assays, the number of surviving cells was also enumerated by determining CFU counts on replica agar plates.

The percentage of VAR6 survival over a period of 5 days and the encapsulation efficacy of VAR6 in the Ag and Ch were determined as described by Oberoi et al. (Oberoi, K. et al., Foods, 2021, 10 (9), 1999. DOI: 10.3390/foods10091999 PubMed). Alg-VAR6-Ch microcapsules were disrupted using 2% (w/v) sterile trisodium citrate solution at pH 6.0, and gently shaken at room temperature for 10 min, followed by sonication on ice for 1 min. The released VAR6 cells were then serially diluted and plated on BHI agar plate and incubated at 37° C. for 24 h and enumerated. Percent encapsulation efficiency and cell survival were calculated as follows:

Encapsulation efficiency (%)=(N/Nₒ)×100

Cell survival (%)=(N/N_{zero})×100

where N is the number of viable encapsulated cells (CFU/ml) released from the microcapsules, and Nₒ is the number of the free viable bacterial cells (CFU/ml) added during the microcapsule preparations for the calculation of encapsulation efficiency. For cell survival, the percentage of VAR6 survival was determined by comparing the number of the viable bacterial cells, N, released from the microcapsules at day 1, 2, 3, 4 and 5 against N_{zero} (number of cells released at day 0). All the experiments were repeated five times and data represent an average of five independent experiments±SD (standard deviation) shown by error bar.

Varying number of VAR6 bacteria were encapsulated in the Alg—1×, 5× and 10× corresponding to 1×10⁹, 5×10⁹ and 1×10¹⁰ CFU/ml, respectively—and their inhibitory activities against M. luteus and S. pyogenes. Table 1 shows that encapsulated microcapsules containing 1×10⁹ CFU/ml of VAR6 yielded the biggest inhibitory zones for both indicator strains, i.e., 3.56 mm for M. luteus and 3.19 mm for S. pyogenes. A concentration of 1×10⁹ CFU/ml of VAR6 was hence used in all subsequent experiments (Table 6).

TABLE 6
Zone of inhibition of Alg-VAR6-Ch microcapsules
against M. luteus and S. pyogenes.

| VAR6 cells | Zone of Inhibition (mm) | |
|---|---|---|
| (cfu/ml) | M. luteus | S. pyogenes |
| 1 × 10⁹ | 3.56 | 3.19 |
| 5 × 10⁹ | 3.00 | 1.50 |
| 10 × 10⁹ | 2.56 | 1.38 |

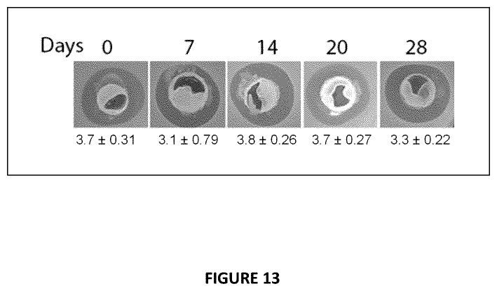

Next, the stability of the inhibitory activity of the Alg-VAR6-Ch was determined over a period of 28 days. As seen in FIG. 13, the inhibitory activities of Alg-VAR6-Ch did not change significantly from days 0 to 28, with measurements of the inhibition zones in the 3.1±0.79 mm (at day 7) to 3.8±0.26 mm (at day 28) range (FIG. 13). This set of data suggests, therefore, that Alg-VAR6-Ch microcapsules, if kept refrigerated, i.e., 4° C., retains the major portion of their inhibitory activities over a period of one month.

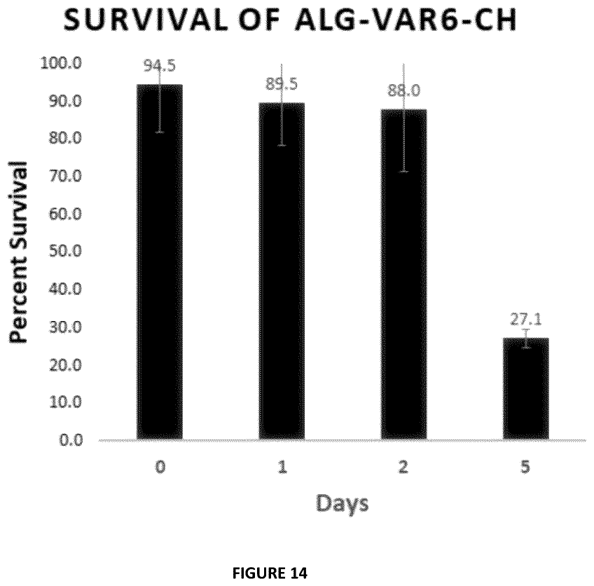

Next, we wanted to determine how long VAR6 survived in its encapsulated form over a 5-day period when stored at 4° C. At day 0, about 94.5% of VAR6 cells were retained in the capsules (FIG. 14). Over the next two days, there was slight decrease in cell survival of 89.5% on day 1 and 88.0% on day 2. By Day 3, however, VAR6 cell viability was reduced to 27.1%. These results suggest that most of the encapsulated VAR6 were no longer viable by day 5. Yet, data from FIG. 1 showed that its inhibitory activities were preserved over a 28-day period (FIG. 13). The combined findings suggest the possibility that antimicrobial peptides were secreted by VAR6 into the Alg encapsulate. Thus, even when VAR6 was no longer viable, the antimicrobial peptides secreted into the Alg were protected and continued to exert their inhibitory effects up to 28 days.

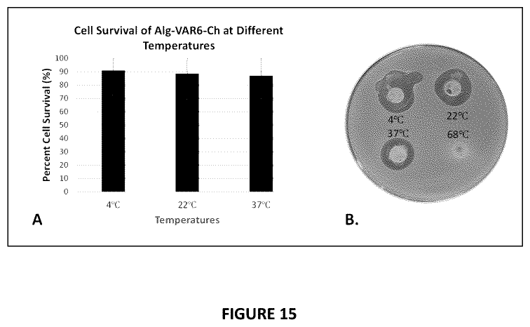

The effect of temperature on the anti-microbial activity of Alg-VAR6 microcapsules was next determined. Four different temperatures were chosen to test the stability of the inhibitory properties of the encapsulated VAR6: 4° C., 22° C. (room temperature), 37° C. (body temperature), and 68° C. (approximate oral cavity temperature during the ingestion of hot beverages). Alg-VAR6-Ch were incubated at these temperatures for 2 h after which they were assayed for cell viability (FIG. 15A) and inhibitory activities (FIG. 15B). Cell viability remained relatively constant from 4° C. (90%) to 22° C. (88%) to 37° C. (87%). At 68° C., no cells were viable (FIG. 15A). This loss of cell viability at 68° C. was also mirrored with a complete loss of inhibitory activities at that temperature (FIG. 15B). From 4° C. to 22° C., the inhibitory zones decreased approximately 10% and a further 3% from 22° C. to 37° C. The lack of viability of Alg-VAR6-Ch at 68° C. is in line what is known about S. salivarius being unable to withstand temperatures of greater than 42° C. Furthermore, it also showed that the antimicrobial peptides were secreted into the Alg encapsulate during the first 2 h.

The study showed the encapsulation of VAR6 in microcapsule form. Ch coating to Alg microcapsules resulted in a significant increase in survival rate and enhanced antibacterial activity of VAR6. In addition, this encapsulating agent enhanced the tolerance of VAR6 to different temperatures without disturbing their antibacterial activity.

### Treatment and Prevention of Periodontal Diseases

VAR6 and SalE4 both inhibit growth of cariogenic biofilms, and a contributor to periodontal diseases is the biofilm formed at the junction of the gum and the tooth. Accordingly, VAR6, SalE4, and oral probiotic/protein-containing products may be useful for the treatment or prevention of periodontal diseases.

The purpose of the above description is to illustrate some configurations and uses of the present invention, without implying any limitation. It will be apparent to those skilled in the art that various modifications and variations may be made in the process and product of the invention without departing from the spirit or scope of the invention.

1. An isolated strain of Streptococcus salivarius having antimicrobial activity against Streptococcus mutans.

2. The isolated strain of Streptococcus salivarius of claim 1, having resistance to deoxyglucose metabolite and/or deletion of manL gene; and/or having stronger adhesion property to biotic and abiotic surfaces; and/or having the ability to form higher biomass.

3. (canceled)

4. (canceled)

5. The isolated strain of Streptococcus salivarius of claim 1, wherein the strain expresses Pep1 having SEQ ID NO.: 5, Pep2 having SEQ ID NO.: 6, Pep3 having SEQ ID NO.: 7, and Pep 4 having SEQ ID NO.: 8.

6. The isolated strain of Streptococcus salivarius of claim 3 expressing the Pep1, Pep2, Pep3 and Pep4 as a multipeptide protein.

7. The isolated strain of Streptococcus salivarius of claim 1, having a 183,700 base pair megaplasmid encoding Pep1, Pep2, Pep3, and Pep4.

8. The isolated strain of Streptococcus salivarius of claim 5, wherein the 183,700 base pair megaplasmid further expresses two ABC transporter ATPase proteins, an ABC transporter permease protein, a histidine kinase, a response regulator, a lantibiotic synthesis protein, and an ABC-type bacteriocin/lantibiotic exporter.

9. The isolated strain of Streptococcus salivarius of claim 1, having inhibitory or toxicity activity against cariogenic bacteria, for example, Streptococcus mutans.

10. (canceled)

11. (canceled)

12. The isolated strain of Streptococcus salivarius of claim 1, having caries prevention activity; and/or having inhibiting activity against streptococcal throat infections; and/or having inhibiting activity against periodontal diseases.

13. (canceled)

14. (canceled)

15. An isolated peptide having at least 90% amino acid similarity or identity to the sequence of SEQ ID No.: 5, SEQ ID NO.: 6, SEQ ID NO.: 7, or SEQ ID NO.: 8 and antimicrobial activity against Streptococcus mutans.

16. An isolated peptide Pep1, Pep2, Pep3, or Pep4, having a sequence of SEQ ID NO.:5, SEQ ID NO.:6, SEQ ID NO.:7, or SEQ ID NO.: 8, respectively.

17. An isolated multipeptide protein comprising peptide Pep1 having a sequence of SEQ ID NO.: 5, peptide Pep2 having a sequence of SEQ ID NO.: 6, peptide Pep3 having a sequence of SEQ ID NO.: 7, and peptide Pep4 having a sequence of SEQ ID NO.: 8.

18. A probiotic mixture comprising the isolated strain of claim 1.

19. A composition for oral administration for preventing caries caused by cariogenic bacteria, such as Streptococcus mutans, comprising the isolated strain of claim 1.

20. A composition for oral administration for preventing or inhibiting cariogenic, e.g., Streptococcus mutans, biofilm formation, comprising the isolated strain of claim 1.

21. A composition for oral administration for prevention or treatment of streptococcal throat infections, comprising the isolated strain of claim 1.

22. A composition for oral administration for prevention or treatment of periodontal diseases comprising the isolated strain of claim 1

23. The probiotic mixture of claim 18, in the form of a lollipop, a candy such as a gummy candy, a lozenge, a mouth wash, a liquid rinse, a toothpaste, a drink, a chewable, a tooth varnish or coating, a lyophilized probiotic powder, or a food.

24. The probiotic mixture of claim 18 in a polysaccharide encapsulated form.

25. The probiotic mixture or composition of claim 24, wherein the polysaccharide encapsulated form is an alginate microcapsule.

26. The probiotic mixture or composition of claim 24, wherein the polysaccharide encapsulated form is a chitosan coated alginate microcapsule.

27. (canceled)

28. (canceled)

29. (canceled)

30. (canceled)

31. (canceled)

32. (canceled)
